(19) United States
(12) Patent Application Publication
(10) Pub. No.: US 2022/0104800 A1
(43) Pub. Date: Apr. 7, 2022

(54) ACCESS NEEDLE SYSTEMS AND METHODS

(71) Applicant: INTERNATIONAL PRIVATE BANK LLC, Christiansted, VI (US)

(72) Inventor: Toufic Kachaamy, Phoenix, AZ (US)

(21) Appl. No.: 17/428,441

(22) PCT Filed: Feb. 3, 2020
(86) PCT No.: PCT/US20/16422

§ 371 Date: Aug. 4, 2021

Related U.S. Application Data
(60) Provisional application No. 62/800,894, filed on Feb. 4, 2019.

(51) Int. Cl. A61B 17/00; A61B 17/34; A61M 5/00; A61B 18/14

(52) U.S. Cl. CPC A61B 17/00234; A61B 17/3403; A61B 17/3417; A61B 17/3468; A61B 2090/3925; A61M 5/007; A61B 18/1482; A61B 17/3478; A61B 17/3496

(57) ABSTRACT

An access needle is provided. The access needle includes a needle housing and a needle shaft having a portion disposed within the needle housing. The access needle also includes a lumen within the needle shaft, an entry port at a proximal end of the needle shaft, and a needle tip at a distal end of the needle shaft. The access needle also includes an exit port on a sidewall of the needle shaft, wherein the exit port is disposed nearer the distal end of the needle shaft than the proximal end, and wherein the lumen extends from the entry port to the exit port. A system and a method for using the access needle are also provided.

### CROSS-REFERENCE TO RELATED APPLICATIONS

The present disclosure is related to, and claims priority under the rules and regulations of the WIPO Patent Cooperation Treaty to U.S. Provisional Patent Application No. 62/800,894 entitled ACCESS NEEDLE SYSTEMS AND METHODS to Toufic Kachaamy, filed on Feb. 4, 2019, the content of which is herein incorporated by reference in their entirety, for all purposes.

### TECHNICAL FIELD

The present description relates in general to medical devices, and more particularly to, for example and without limitation, access needles and methods and uses thereof.

### BACKGROUND OF THE DISCLOSURE

An estimated 1,230,000 endoscopic retrograde cholangiopancreatography (“ERCP”) procedures were performed in the 28 member countries of the European Union and the United States in 2016. As part of an ERCP procedure, cannulation must first be achieved in order to gain access to the desired duct(s); however, this can sometimes be challenging. One approach is to use a sphincterotome device (also called a papillotome), inserted through a working channel of a duodenoscope. A sphincterotome is a catheter that contains an electrosurgical cutting wire at the distal end, which is used to perform sphincterotomies (e.g., cutting of sphincter muscles in order to gain duct access to perform follow-up procedures). However, in some scenarios, ERCP can fail due to cannulation failure, even using a sphincterotome in some cases, and/or due to inability to access the papilla.

The description provided in the background section should not be assumed to be prior art merely because it is mentioned in or associated with the background section. The background section may include information that describes one or more aspects of the subject technology.

### SUMMARY

In one embodiment, an access needle is provided that includes a needle housing and a needle shaft having a portion disposed within the needle housing. The needle also includes a lumen within the needle shaft, an entry port at a proximal end of the needle shaft, and a needle tip at a distal end of the needle shaft. The needle also includes an exit port on a sidewall of the needle shaft, wherein the exit port is disposed nearer the distal end of the needle shaft than the proximal end, and wherein the lumen extends from the entry port to the exit port.

In a second embodiment, a method is provided that includes placing a needle in a vicinity of an organ, the organ comprising a target portion. The needle comprises: a needle housing, a needle shaft, a lumen within the needle shaft, an entry port at a proximal end of the needle shaft, a needle tip at a distal end of the needle shaft, and an exit port on a sidewall of the needle shaft. The method also includes piercing the organ with the needle tip, and injecting a contrast medium into the organ through the lumen in the needle shaft. The method also includes adjusting a position of the needle in the organ based on an image of the organ, the image comprising the contrast agent, so that the exit port points to the target portion, and inserting a wire through the entry port, the lumen, and the exit port, to access the target portion.

In yet other embodiment, a system, a system is provided that includes an access needle to direct a guiding element to a target portion of an organ, the access needle including a needle housing and a needle shaft having a portion disposed within the needle housing. The access needle further includes a lumen within the needle shaft, an entry port at a proximal end of the needle shaft and a needle tip at a distal end of the needle shaft. The access needle also includes an exit port on a sidewall of the needle shaft, wherein the exit port is disposed nearer the distal end of the needle shaft than the proximal end, and wherein the lumen extends from the entry port to the exit port. The system also includes an imaging device to direct the access needle in a vicinity of the organ and puncture an access site in the organ with the needle tip. The imaging device further includes a radiation source configured to direct a radiation to the organ, and a radiation detector configured to detect a scattered radiation from the organ. The imaging device also includes a controller, configured to receive a signal from the radiation detector and convert the signal into an image and a display, configured to display the image from the controller to direct the needle in the vicinity of the organ.

### BRIEF DESCRIPTION OF THE DRAWINGS

FIG. 1 illustrates a diagram of a biliary tree structure.

FIG. 2 illustrates a diagram of a part of an access needle, in accordance with various aspects of the subject disclosure.

FIG. 3 illustrates an enlarged cross-sectional view of a distal end of a needle shaft of the access needle of FIG. 2, in accordance with various aspects of the subject disclosure.

FIG. 4 illustrates an enlarged cross-sectional view of a distal end of a needle shaft of the access needle of FIG. 2 with a wire disposed in a lumen of the shaft, in accordance with various aspects of the subject disclosure.

FIG. 5 illustrates an enlarged cross-sectional view of a portion of a needle shaft of the access needle of FIG. 2 in the vicinity of a lock, in accordance with various aspects of the subject disclosure.

FIG. 6 illustrates an enlarged cross-sectional view of a distal end of a needle shaft of the access needle of FIG. 2 disposed within a bile duct, in accordance with various aspects of the subject disclosure.

FIG. 7 illustrates an enlarged cross-sectional view of a distal end of a needle shaft of the access needle of FIG. 2 disposed within a bile duct and rotated with respect to the orientation shown in FIG. 6, in accordance with various aspects of the subject disclosure.

FIG. 8 illustrates a diagram of an access needle having a sheath, in accordance with various aspects of the subject disclosure.

FIG. 9 illustrates a portion of the access needle of FIG. 8, including a tapered portion of the sheath, in accordance with various aspects of the subject disclosure.

FIG. 10 illustrates an electrocautery enhanced access needle to deliver a cautery current to treat bleeding along an access site, in accordance with various aspects of the subject disclosure.

FIG. 11 illustrates an access needle including a rotatable mechanism to guide a wire to a target portion in an organ, in accordance with various aspects of the subject disclosure.

FIG. 12 illustrates an access needle accessing a target portion of an organ during surgery of a patient, in accordance with various aspects of the subject disclosure.

FIG. 13 is a flowchart illustrating steps in a method for accessing a target portion of an organ during surgery of a patient, in accordance to various embodiments.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

Access of a duct or cavity with endoscopic ultrasound (EUS) has become more common, such as for bile duct drainage when ERCP fails or is not possible. This often requires access of the duct or cavity with a needle under EUS guidance, and then passing a wire to allow for further passage of other tools over the wire into the cavity. This technique can be used for gallbladder access and drainage when patients have a gallbladder infection and surgery is not desired, and for drainage of fluids around the gastrointestinal track such as drainage of collections around the pancreas or the rectum.

Currently endoscopic ultrasound-guided (EUS-guided) biliary access is considered a rescue technique when endoscopic retrograde cholangiography (ERCP) fails due to cannulation failure or the inability to access the papilla. Endoscopic ultrasound-guided biliary access, however, is very technically challenging. One of the reasons for the technically challenging nature of this procedure is the lack of dedicated tools, specifically tools that facilitate biliary access and allow manipulation of a wire in a desired direction, either to perform rendezvous (e.g., in which the wire is passed down the papilla and then captured to be used to facilitate standard ERCP), or to advance the wire toward the liver and perform direct stenting of the bile duct.

Access needles for biliary access are disclosed herein. Access needles are also used to access the gallbladder, and abscesses and collections next to the gastrointestinal tract, in addition to uses for pancreatic cyst and pancreatic duct access. Some access needles include a 19-gauge access needle making them stiff and difficult to manipulate and advance into the bile duct. Moreover, these access needles typically include an opening for wire access at the tip of the needle. An opening for wire access at the tip of the needle creates a risk of wire shearing by the tip. The risk of wire shearing can be reduced by providing a pointed cutting tip on a stylet that is removed once the access is achieved, allowing insertion and manipulation of the wire. However, even with the reduced risk of shearing provided by a stylet, access needles with the wire opening at the tip do not allow easy guidance of the wire direction, and some risk of shearing may still remain.

The common biliary duct splits into left and right hepatic ducts, and an exemplary access needle of the present disclosure may help with accessing these ducts more easily. In some procedures, high obstruction of the biliary tree may require the placement of two guidewires. An example embodiment of the present disclosure allows two or more wires to be placed in selected dilated ducts for faster access, thus shortening the procedure. In some embodiments of the present disclosure, an exemplary access needle makes the placement of two wires easier to perform, by allowing rotation of the location of a wire access port on a side of the needle.

In accordance with aspects of the subject disclosure, an improved access needle is provided. The improved access needle may be smaller than currently available needles and can vary in size (e.g., between about 25-gauge and about 18-gauge, such as about 22-gauge), so as to be easier to manipulate and advance into the bile duct.

The needle includes an internal lumen that extends between a wire entry port at a proximal end and a wire exit port on a sidewall of the needle. The lumen may include guide features that guide the wire from the lumen to the exit port along a curve that is not sharp. In this way, the wire exit port does not cause shearing of the wire, forms a wide exit at an angle at a location that is not from the tip of the needle, and facilitates rotation of the direction of exit of the wire from the port. For example, the needle can be rotatable within and/or by a needle housing to rotate the location of the wire exit port. In this way, the disclosed access needle allows the wire to be manipulated to exit in any desired direction. The needle shaft that includes the lumen may be formed from a flexible material, and includes a tip shaped to facilitate advancing the needle and cutting through tough tissue such as the bile duct.

Accordingly, embodiments of the present disclosure may achieve advantages such as wire access to the bile duct for patients with challenging ductal anatomies.

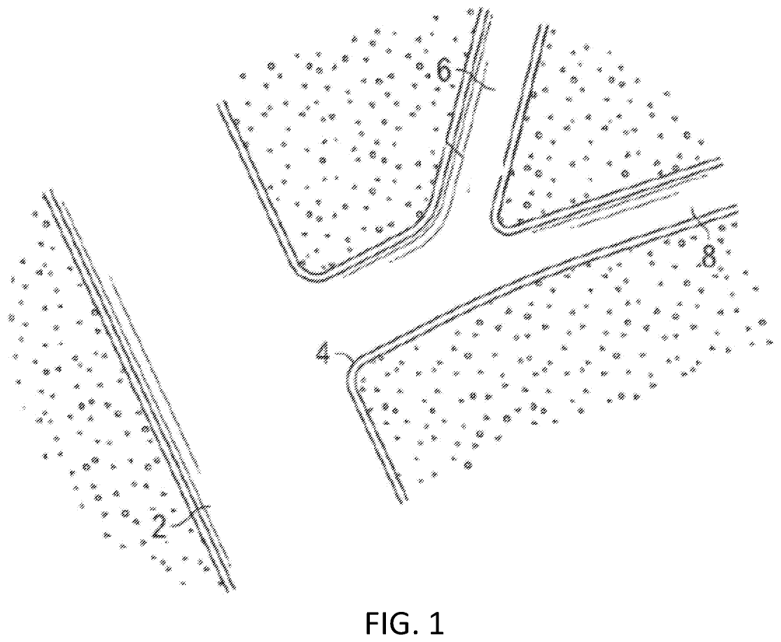

FIG. 1 illustrates a portion of the duodenum 2 at the location of a sphincter 4 (i.e., sphincter of Oddi) at the major duodenal papilla. The biliary duct 6 and pancreatic duct 8 are also shown in FIG. 1.

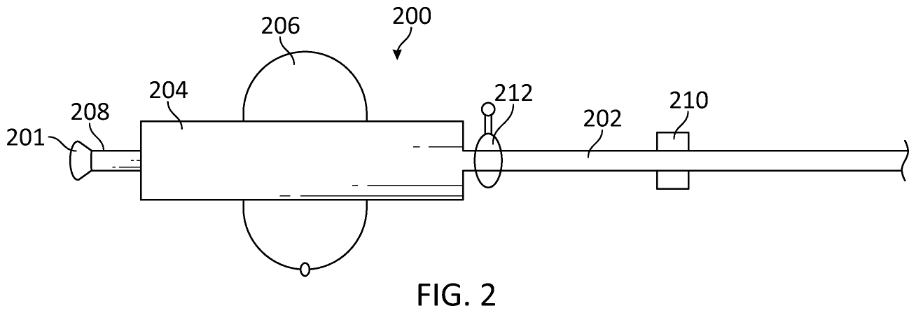

FIG. 2 illustrates a side view of an example access needle 200 that may be used to access, for example, biliary duct 6 (e.g., for insertion of a wire such as a guide wire). Access needle 200 includes a needle housing 204 in which a needle shaft 202 is disposed. A wire entry port 201 is an opening to allow entering of the wire at the proximal end of needle housing 204. Needle shaft 202 may be fixedly disposed in needle housing 204 such that needle shaft 202 can be advanced and/or rotated by advancing and/or rotating the housing through a rotatable handle 206. For example, the needle shaft may be fixed to the housing by a locking mechanism 212 or can be permanently fixed to the housing. Alternatively, the needle shaft may be moveably disposed in the needle housing such that the needle shaft can be advanced and/or rotated with respect to the housing. As shown in FIG. 2, access needle 200 may also include an entry port 201 at a proximal end 208 of the needle shaft, a handle 206 on the needle housing 204 (e.g., for rotating and advancing the needle), a lock 210 for locking the needle shaft 202 to an endoscope channel, and locking mechanism 212. Locking mechanism 212 may be used for locking the needle shaft in place relative to the needle housing 204 and/or locking the needle shaft 202 to another object.

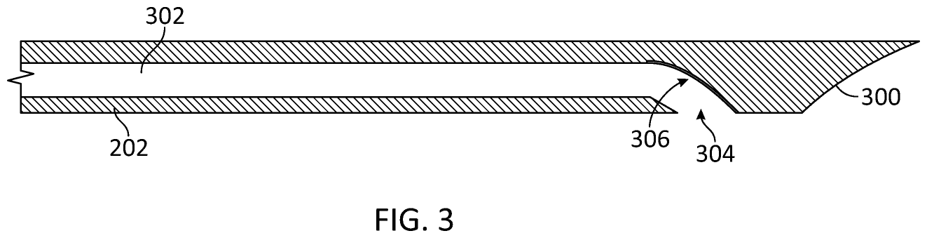

FIG. 3 shows an enlarged cross-sectional view of the distal end of needle shaft 202. As shown in FIG. 3, needle shaft 202 includes a needle tip 300 at a distal end of the needle shaft, and a lumen 302 within the needle shaft. An exit port 304 is also shown on a sidewall of the needle shaft 202, nearer the distal end of the needle shaft than the proximal end. Exit port 304 enables the wire to leave the needle shaft 202 and access the biliary duct or other organ portion. In various examples, needle shaft 202 has a gauge of between about 25 and about 18 (e.g., about twenty-two or higher). FIG. 3 also shows how needle shaft 202 may include a guide surface 306 that is gently curved and thus configured to guide a wire from the lumen 302 through the exit port 304. In accordance to various embodiments, guide surface 306 may have a smooth texture to decrease the risk of shearing, stopping, or jerking the wire as it passes through and is bent onto exit port 304. For example, in some embodiments, the system may include a stylet configured to cover guide surface 306 and make it smooth.

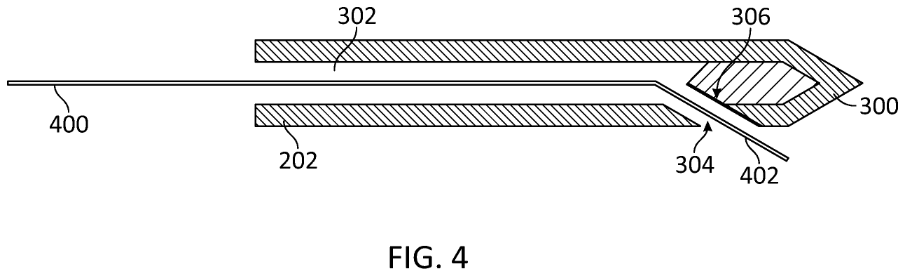

FIG. 4 illustrates an arrangement in which a wire 400 is disposed within lumen 302, and in which distal portion 402 of wire 400 has been guided by guide surface 306 from the lumen out of the side of needle shaft 202 via exit port 304, proximal to needle tip 300. In various scenarios, a second wire may also be fed through the lumen 302, e.g., through a second entry port at the proximal end of the needle shaft to receive the second wire.

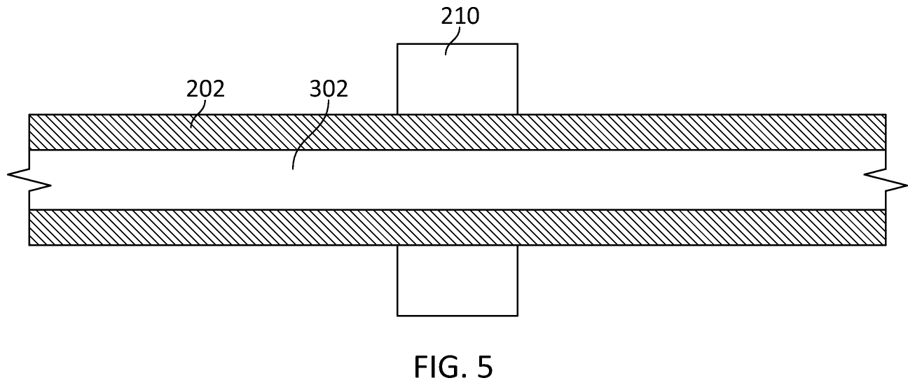

FIG. 5 illustrates a further enlarged cross-sectional view of a portion of needle shaft 202 forming lumen 302 in the vicinity of lock 210.

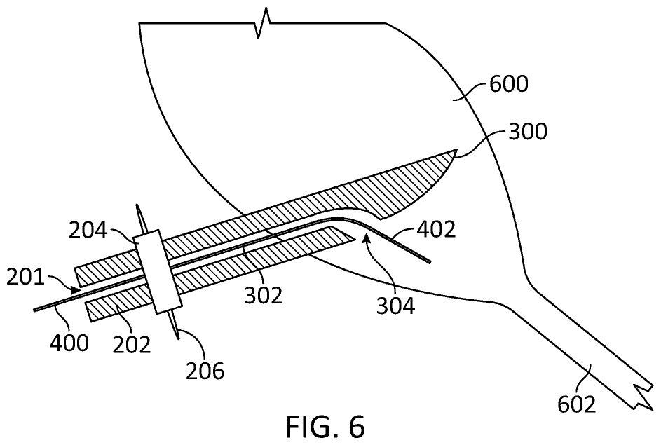

FIG. 6 illustrates an arrangement in which the tip 300 of needle shaft 202 has pierced an organ 600 (e.g., duct 6 of FIG. 1 in a dilated state) above a target portion (e.g., an obstruction 602), and in which wire 400 has been inserted into the bile duct in the direction of the obstruction via exit port 304 in the side of needle shaft 202. Because needle shaft 202 (or a portion thereof such as the distal portion) is rotatable, the exit port 304 can be positioned and oriented such that distal portion 402 of wire 400 exits the needle in the desired direction.

To achieve the arrangement shown in FIG. 6, needle shaft 202 may be advanced until tip 300 pierces the bile duct 600. The bile duct may then be aspirated and/or injected with contrast media, such as a contrast dye, through the needle to verify the location of the needle. Needle shaft 202 and exit port 304 in the sidewall of the needle shaft may then be rotated to a desired location and/or orientation. A wire such as wire 400 may then be inserted via entry port 201, through lumen 302 and out through exit port 304. Needle housing 204 and handle 206 are also illustrated, for perspective.

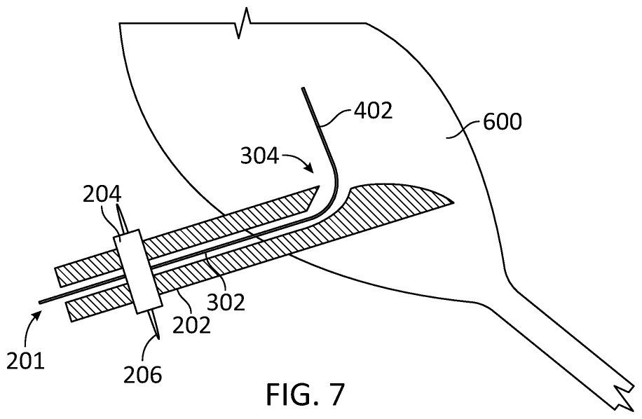

FIG. 7 illustrates an example in which exit port 304 has been rotated such that distal portion 402 of wire 400 exits in the opposite direction from that shown in FIG. 6. Exit port 304 can be rotated by rotating the distal portion of needle shaft 202, by rotating the entirety of needle shaft 202, and/or by rotating housing 204 with handle 206. Also illustrated are entry port 201, lumen 302, and organ 600.

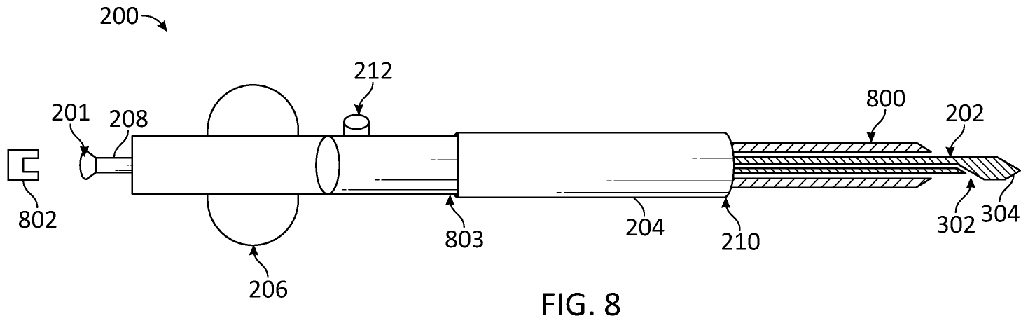

FIG. 8 shows a side view of access needle 200 in an alternative implementation in which a sheath 800 is formed around at least a portion of needle shaft 202. Sheath 800 may be formed from plastic, metal, or other biocompatible materials and can be softer and/or more flexible than the metal of needle shaft 202. In the example of FIG. 8, sheath 800 is positioned such that at least some of the sheath 800 is disposed between the needle housing 204 and the needle shaft 202.

In this example, handle 206 for rotation of the position of exit port 304 is disposed on a proximal side of needle housing 204. In this example, locking mechanism 212 is also disposed on the proximal side of needle housing 204 and is configured to lock the needle shaft 202 in position relative to the sheath 800. For example, when locking mechanism 212 locks needle shaft 202 in position relative to sheath 800, handle 206 can be manipulated to rotate sheath 800 within, or along with, needle housing 204 to rotate needle shaft 202 and consequently exit port 304. When locking mechanism 212 is unlocked, needle shaft 202 may be rotatable and/or slidable within sheath 800. In this example, lock 210 may lock access needle 200 to an endoscope such that needle shaft 202 remains movable (e.g., slidable) with respect to the tip of the endoscope within or along with sheath 800.

In one arrangement, access needle 200 is configured to allow adjustment of the position of the needle shaft 202 relative to the sheath 800 and to allow advancement of the sheath 800 over the needle shaft 202 if desired, allowing duct and cavity access via the sheath 800. In the example of FIG. 8, needle 200 is shown in side view, with the distal portion of sheath 800 shown in cross-section for clarity. As indicated in FIG. 8, needle 200 may include features 803 for controlling the position and movement of needle shaft 202 relative to the sheath 800.

In the example of FIG. 8, at least one of the needle housing 204, the sheath 800, or the needle shaft 202 is rotatable (e.g., together or separately) for modification of an exit direction of a wire in the lumen 302 from the exit port 304 by rotation of the exit port. In this example, the needle shaft 202 may be slidably disposed within the sheath 800 (e.g., when locking mechanism 212 is unlocked). In the example of FIG. 8, a removable cover 802 is provided for the entry port 201 near proximal end 208. Cover 802 may also be configured to removably cover entry port 201 of FIG. 2. In the examples of FIGS. 2 and 8, entry port 201 is configured to receive a wire for passage of the wire through the lumen 302 and the exit port 304. Entry port 201 may also include an attachment feature (not explicitly shown) for attachment of a syringe to provide fluid into the lumen 302 to the exit port 304. In implementations in which sheath 800 is provided, the sheath may have a tapered distal end.

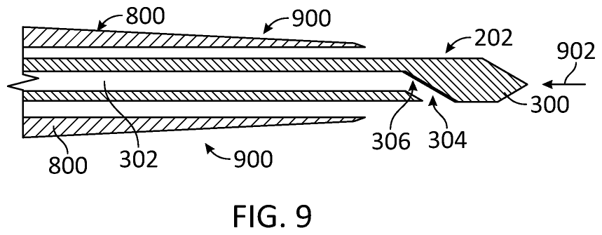

FIG. 9 shows a portion of the access needle of FIG. 8 in an example in which sheath 800 includes a tapered portion 900. In this example, sheath 800 has a proximal end (not shown in FIG. 9) and a distal end, and the distal end includes the tapered portion 900, which tapers toward the cutting tip 300 of the needle shaft 202. In this example, the sheath 800 is tapered at the distal end to, for example, allow advancing of sheath 800 over the needle shaft 202 (e.g., by movement of needle shaft 202 relative to sheath 800 parallel to direction 902) into the cavity or duct to be used as an access device if the needle shaft 202 is removed. In one operational scenario, a first wire can be inserted into the duct via lumen 302 in needle shaft 202, sheath 800 can be advanced over the needle shaft into the duct, needle shaft 202 can be removed, and a larger wire can be fed into the duct along the first wire through the relatively wider lumen of the sheath. Guide surface 306 bends the wire and directs it towards exit port 304.

It is understood that the specific order or hierarchy of steps, operations, or processes disclosed is an illustration of exemplary approaches. Unless explicitly stated otherwise, it is understood that the specific order or hierarchy of steps, operations, or processes may be performed in different order. Some of the steps, operations, or processes may be performed simultaneously. The accompanying method claims, if any, present elements of the various steps, operations, or processes in a sample order, and are not meant to be limited to the specific order or hierarchy presented. These may be performed in serial, linearly, in parallel, or in different order.

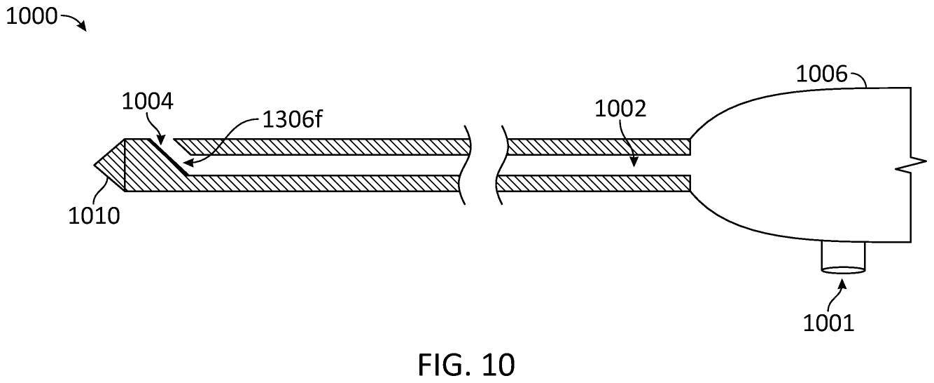

FIG. 10 illustrates an electrocautery enhanced access needle 1000 to deliver a cautery current to treat bleeding along an access site, in accordance with various aspects of the subject disclosure. A handle 1006 may be used to adjust (e.g., rotate, pitch, and roll) the position of electrocautery enhanced needle 1000 inside an organ and to point exit port 1004 in a desired direction (e.g., a target portion of the organ).

A port 1001 in handle 1006 receives an electric current and transmits the electric current to tip 1010. In some embodiments, tip 1010 includes an electrically conductive material while the rest of the shaft is shielded from electricity. Tip 1010 thus delivers the current into the surrounding tissue in the organ. In some embodiments, the current acts as a cautery current to treat bleeding along the access site of the organ (e.g., the point at which the tip has punctured the organ) by allowing blood coagulation through heat. In some embodiments, the current acts as a cutting element to dilate the access site of the organ and facilitate insertion of other devices in the organ, through the enlarged access site. Electrocautery enhanced access needle 1000 also includes a guiding surface 1306f to guide a wire or any other guiding component through lumen 1002 out of exit port 1004 (e.g., guiding surface 306, lumen 202, and exit port 304). In some embodiments, the electrocautery tip is on the shaft of the needle and can be used to treat bleeding or to allow advancement of the shaft over a wire or the needle into the tissue or duct.

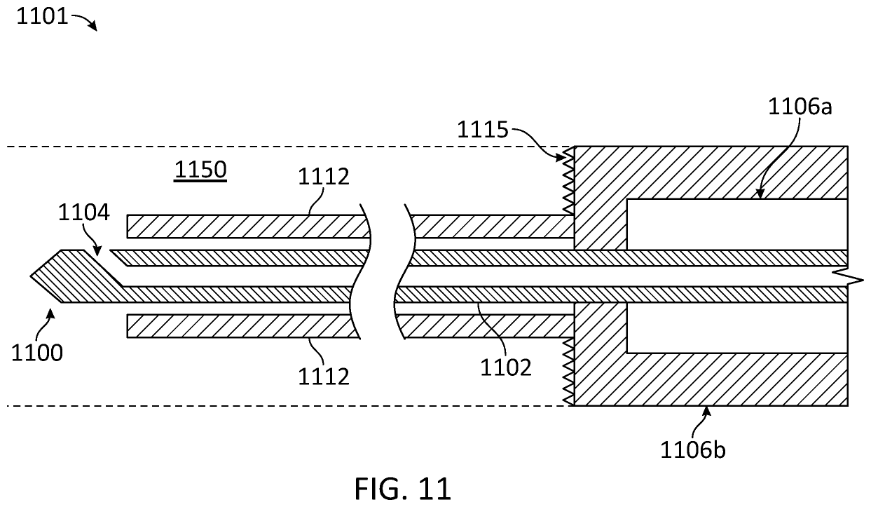

FIG. 11 illustrates an access needle 1101 including a rotatable mechanism to guide a wire to a target portion in an organ, in accordance with various aspects of the subject disclosure. Access needle 1101 is housed longitudinally (along the axis of the channel) inside a sheath 1112 (e.g., outer catheter). Access needle 1101 is coupled with an inner handle 1106a. Inner handle 1106a is housed inside an outer handle 1106b. Outer handle 1106b may be coupled with a biopsy channel 1150 via an anchoring mechanism 1115 (e.g., screw on, for fixation to a scope—not shown in the figure—). Inner handle 1106a rotates inside from and relative to, outer handle 1106b, about a common longitudinal axis. Rotation of inner handle 1106a leads to rotation of access needle 1101 including needle tip 1100, needle shaft 1102, and exit port 1104. The rotation gives the ability to guide the direction of a wire towards the target portion of the organ (e.g., liver or duodenum). Note that, according to some embodiments, while needle shaft 1102 rotates about a longitudinal axis, sheath 1112 remains stationary, thus reducing friction with exterior tissue or other endoscopic components.

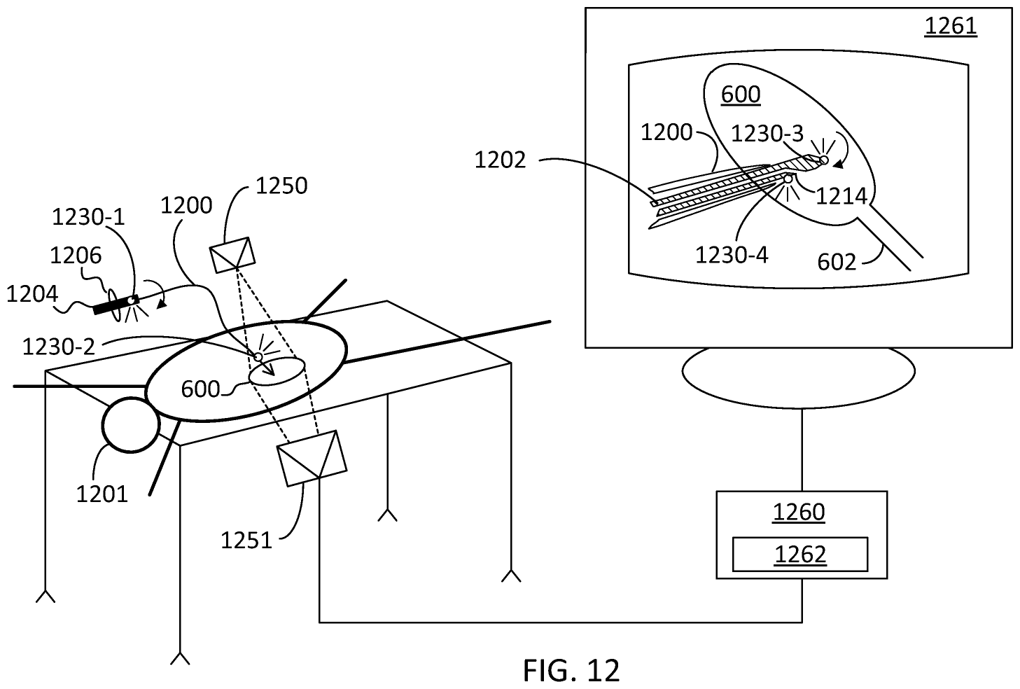

FIG. 12 illustrates an access needle 1200 accessing a target portion 602 (e.g., bile duct obstruction, and the like) of an organ 600 during surgery of a patient 1201, in accordance with various aspects of the subject disclosure. In some embodiments, an imaging system 1260 may be used as an aid tool during surgery. Imaging system may include a radiation source 1250 (e.g., an ultrasound source, a light source, an X-ray source, and the like) emitting a radiation that is scattered from at least a portion of organ 600 and collected by a radiation detector 1251 (e.g., an ultrasound detector, a light detector, an X-ray detector, and the like). Imaging system 1260 reads a signal from radiation detector 1251 and generates an image of the surgical area in display 1261. To do these operations, imaging system 1260 includes a controller 1262 that performs analog and digital electronic data analysis on the signal provided by radiation detector 1251. In the image, the surgeon or nurse may have an augmented and clear view of access needle 1200, organ 600, and target portion 602. Accordingly, the surgeon or nurse may assess the relative position and orientation of exit port 1214 and target portion 602. Thus, the surgeon or nurse may actuate a handle 1206 to adjust the position of access needle 1200 in organ 600.

In some embodiments, access needle 1200 may include one or more markers 1230-1, 1230-2, 1230-3, and 1230-4 (hereinafter, collectively referred to as “markers 1230”) disposed adjacent to at least one of the needle tip, exit port 1214, a needle housing 1204, a needle shaft 1202, or handle 1206. In some embodiments, markers 1230 are configured to scatter an ultrasound radiation, an electromagnetic radiation, or any other type of radiation or combination thereof. In some embodiments, markers 1230 may be detectable with radiation detector 1251.

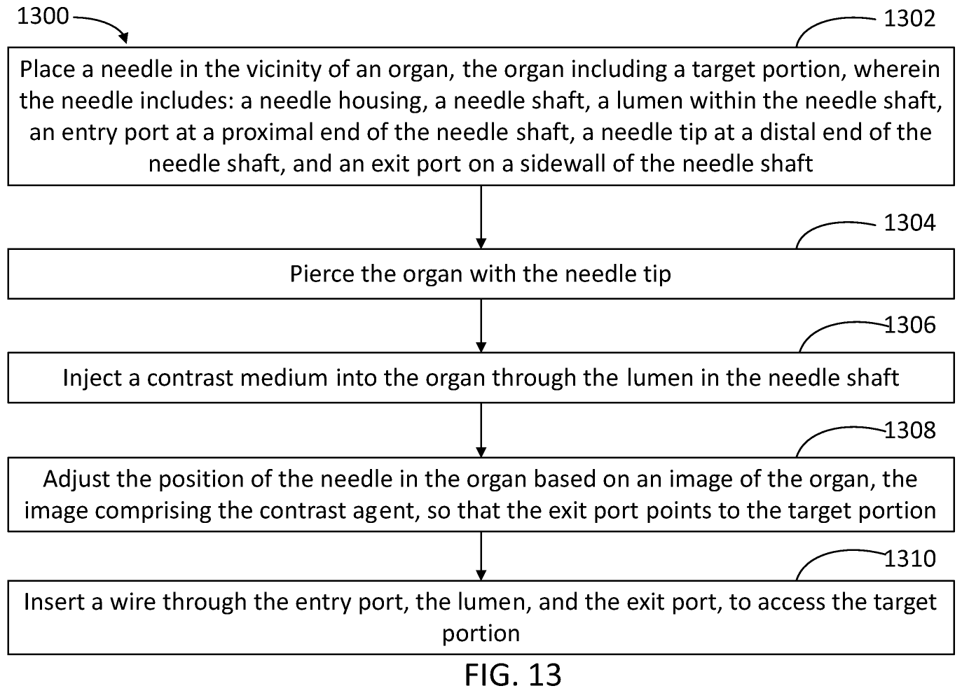

FIG. 13 is a flowchart illustrating steps in a method 1300 for accessing a target portion of an organ during surgery of a patient using an imaging system (e.g., target portion 602 in organ 600 of patient 1201 and imaging system 1260, cf. FIG. 12), in accordance to various embodiments. Methods and systems consistent with the present disclosure may include at least one or more of the steps in method 1300 performed in the same or different order. For example, in some embodiments, a method consistent with the present disclosure may include one or more of the steps in method 1300 performed simultaneously, quasi-simultaneously, or overlapping in time.

Step 1302 includes placing a needle in a vicinity of the organ, the organ including the target portion (e.g., electrocautery enhanced access needle 1000, and access needle 1200, cf. FIGS. 1-11). The needle includes a needle housing, a needle shaft, a lumen within the needle shaft, an entry port at a proximal end of the needle shaft, a needle tip at a distal end of the needle shaft, and an exit port on a sidewall of the needle shaft (e.g., housing 204, cutting tip 300, needle shaft 202, handle 206, locking mechanism 212, entry port 201, exit port 304, lumen 302, and wire 400, cf. FIGS. 1-10).

Step 1304 includes piercing the organ with the needle tip. In some embodiments, step 1304 includes directing an electric current through the tip into an access point in the organ to dilate the access point in the organ.

Step 1306 includes injecting a contrast medium into the organ through the lumen in the needle shaft.

Step 1308 includes adjusting a position of the needle in the organ based on an image of the organ, the image including the contrast agent, so that the exit port points to the target portion. In some embodiments, step 1308 includes rotating the needle housing along a longitudinal axis in the needle shaft. In some embodiments, step 1308 includes directing a radiation to the organ and collecting a scattered radiation from the organ with an imaging system.

Step 1310 includes inserting a wire through the entry port, the lumen, and the exit port, to access the target portion. In some embodiments, step 1310 includes removing the needle housing and the needle shaft and guiding a medical device through the wire, to the target portion. In some embodiments, the target portion is an obstructed duct, and step 1310 includes removing the needle housing and the needle shaft and guiding a stent through the wire to the obstructed duct. In some embodiments, step 1310 includes directing an electric current through the needle tip into an access point in the organ, to cauterize a bleeding or create a path for passing larger devices or instruments.

A reference to an element in the singular is not intended to mean one and only one unless specifically so stated, but rather one or more. For example, “a” module may refer to one or more modules. An element proceeded by “a,” “an,” “the,” or “said” does not, without further constraints, preclude the existence of additional same elements.

Headings and subheadings, if any, are used for convenience only and do not limit the invention. The word exemplary is used to mean serving as an example or illustration. To the extent that the term include, have, or the like is used, such term is intended to be inclusive in a manner similar to the term comprise, as comprise is interpreted when employed as a transitional word in a claim. Relational terms such as first and second and the like may be used to distinguish one entity or action from another without necessarily requiring or implying any actual such relationship or order between such entities or actions.

Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A phrase “at least one of” preceding a series of items, with the terms “and” or “or” to separate any of the items, modifies the list as a whole, rather than each member of the list. The phrase “at least one of” does not require selection of at least one item; rather, the phrase allows a meaning that includes at least one of any one of the items, and/or at least one of any combination of the items, and/or at least one of each of the items. By way of example, each of the phrases “at least one of A, B, and C” or “at least one of A, B, or C” refers to only A, only B, or only C; any combination of A, B, and C; and/or at least one of each of A, B, and C.

In one aspect, a term coupled or the like may refer to being directly coupled. In another aspect, a term coupled or the like may refer to being indirectly coupled.

Terms such as top, bottom, front, rear, side, horizontal, vertical, distal, proximal, and the like refer to an arbitrary frame of reference, rather than to the ordinary gravitational frame of reference. Thus, such a term may extend upwardly, downwardly, diagonally, or horizontally in a gravitational frame of reference.

The disclosure is provided to enable any person skilled in the art to practice the various aspects described herein. In some instances, well-known structures and components are shown in block diagram form in order to avoid obscuring the concepts of the subject technology. The disclosure provides various examples of the subject technology, and the subject technology is not limited to these examples. Various modifications to these aspects will be readily apparent to those skilled in the art, and the principles described herein may be applied to other aspects.

All structural and functional equivalents to the elements of the various aspects described throughout the disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

### RECITATION OF EMBODIMENTS

Embodiments disclosed herein include:

I. An access needle including a needle housing is provided. The access needle includes a needle shaft having a portion disposed within the needle housing, a lumen within the needle shaft, and an entry port at a proximal end of the needle shaft. The access needle also includes a needle tip at a distal end of the needle shaft, and an exit port on a sidewall of the needle shaft, wherein the exit port is disposed nearer the distal end of the needle shaft than the proximal end, and wherein the lumen extends from the entry port to the exit port.

II. A method for placing a needle in a vicinity of an organ, the organ including a target portion, is provided. The needle includes: a needle housing, a needle shaft, a lumen within the needle shaft, an entry port at a proximal end of the needle shaft, a needle tip at a distal end of the needle shaft, and an exit port on a sidewall of the needle shaft. The method includes piercing the organ with the needle tip, injecting a contrast medium into the organ through the lumen in the needle shaft, adjusting a position of the needle in the organ based on an image of the organ, the image including the contrast agent, so that the exit port points to the target portion, and inserting a wire through the entry port, the lumen, and the exit port, to access the target portion.

III. A system including an access needle to direct a guiding element to a target portion of an organ is provided. The access needle includes a needle housing, a needle shaft having a portion disposed within the needle housing, a lumen within the needle shaft, and an entry port at a proximal end of the needle shaft. The access needle also includes a needle tip at a distal end of the needle shaft, and an exit port on a sidewall of the needle shaft, wherein the exit port is disposed nearer the distal end of the needle shaft than the proximal end, and wherein the lumen extends from the entry port to the exit port. The system includes an imaging device to direct the access needle in a vicinity of the organ and puncture an access site in the organ with the needle tip. The imaging device includes a radiation source configured to direct a radiation to the organ, a radiation detector configured to detect a scattered radiation from the organ, a controller, configured to receive a signal from the radiation detector and convert the signal into an image, and a display, configured to display the image from the controller to direct the needle in the vicinity of the organ.

Additionally to embodiments I, II, and III, embodiments consistent with the present disclosure may include any one or more of the following elements, in any combination.

Element 1, wherein the needle shaft has a gauge of about twenty-five or higher. Element 2, further including a guide surface configured to guide a wire from the lumen through the exit port. Element 3, further including a wire in the lumen. Element 4, further including a second wire in the lumen, and a second entry port at the proximal end of the needle shaft to receive the second wire. Element 5, further including a locking mechanism. Element 6, further including a lock for attaching the needle shaft to an endoscope channel of an endoscope. Element 7, wherein the needle shaft is adjustable within the needle housing to allow movement of the needle shaft relative to a tip of the endoscope. Element 8, wherein at least one of the needle housing, the needle shaft, or the distal end of the needle shaft is rotatable for modification of an exit direction of a wire from the exit port, by rotation of the exit port. Element 9, further including a sheath disposed around the needle shaft. Element 10, further including a locking mechanism configured to lock the needle shaft in position relative to the sheath. Element 11, wherein at least one of the needle housing, the sheath, the needle shaft, or the distal end of the needle shaft is rotatable for modification of an exit direction of a wire from the exit port, by rotation of the exit port. Element 12, wherein the needle shaft is slidably disposed within the sheath. Element 13, wherein the sheath has a proximal end and a distal end, and wherein the distal end tapers toward the needle tip of the needle shaft. Element 14, further including a removable cover for the entry port. Element 15, wherein the entry port is configured to receive a wire for passage of the wire through the lumen and the exit port. Element 16, wherein the entry port further includes an attachment feature for attachment of a syringe to provide fluid into the lumen to the exit port. Element 17, wherein the lumen forms a guiding surface to bend the wire to the exit port within the needle shaft. Element 18, further including one or more markers disposed adjacent to at least one of the needle tip or the exit port, the one or more markers being detectable with an ultrasound radiation or an electromagnetic radiation. Element 19, further including one or more markers disposed adjacent to at least one of the needle housing, the needle shaft, or a needle handle, the one or more markers configured to scatter an ultrasound radiation or an electromagnetic radiation.

Element 20, wherein piercing the organ with the needle tip further includes directing an electric current through the needle tip into an access point in the organ to dilate the access point in the organ. Element 21, wherein adjusting the position of the needle in the organ includes rotating the needle housing about a longitudinal axis in the needle shaft. Element 22, wherein adjusting the position of the needle in the organ includes directing a radiation to the organ and collecting a scattered radiation from the organ with an imaging system. Element 23, further including removing the needle housing and the needle shaft and guiding a medical device through the wire, to the target portion. Element 24, wherein the target portion is an obstructed duct, further including: removing the needle housing and the needle shaft; and guiding a stent through the wire to the obstructed duct. Element 25, further including directing an electric current through the needle tip into an access point in the organ, to cauterize a bleeding. Element 26, wherein adjusting a position of the needle in the organ based on an image of the organ includes receiving an ultrasound radiation or an electromagnetic radiation scattered from one or more markers disposed adjacent to at least one of the needle tip or the exit port, and including an image of the needle in the image of the organ using the ultrasound radiation or the electromagnetic radiation. Element 27, wherein adjusting a position of the needle in the organ based on an image of the organ includes receiving an ultrasound radiation or an electromagnetic radiation scattered from one or more markers disposed adjacent to at least one of the needle housing, the needle shaft, or a needle handle, and including an image of the needle in the image of the organ based on the ultrasound radiation or the electromagnetic radiation.

Element 28, wherein the needle shaft has a gauge of about twenty-five or higher. Element 29, further including a guide surface configured to guide a wire from the lumen through the exit port. Element 30, further including a wire in the lumen. Element 31, further including a locking mechanism. Element 32, further including a lock for attaching the needle shaft to an endoscope channel of an endoscope, wherein the needle shaft is adjustable within the needle housing to allow movement of the needle shaft relative to a tip of the endoscope. Element 33, wherein at least one of the needle housing, the needle shaft, or the distal end of the needle shaft is rotatable for modification of an exit direction of a wire from the exit port, by rotation of the exit port. Element 34, further including a sheath disposed around the needle shaft, and a locking mechanism configured to lock the needle shaft in position relative to the sheath, wherein at least one of the needle housing, the sheath, the needle shaft, or the distal end of the needle shaft is rotatable for modification of an exit direction of a wire from the exit port, by rotation of the exit port, wherein the needle shaft is slidably disposed within the sheath, and wherein the sheath has a proximal end and a distal end, and wherein the distal end tapers toward the needle tip of the needle shaft. Element 35, further including a removable cover for the entry port. Element 36, wherein the entry port is configured to receive a wire for passage of the wire through the lumen and the exit port, wherein the entry port further includes an attachment feature for attachment of a syringe to provide fluid into the lumen to the exit port, and wherein the lumen forms a guiding surface to bend the wire to the exit port within the needle shaft. Element 37, wherein the access needle further includes one or more markers disposed adjacent to at least one of the needle tip, the exit port, the needle housing, the needle shaft, or a needle handle, the one or more markers configured to scatter an ultrasound radiation or an electromagnetic radiation generated by the radiation source.

The title, background, brief description of the drawings, and drawings are hereby incorporated into the disclosure and are provided as illustrative examples of the disclosure, not as restrictive descriptions. They are submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirements of the applicable patent law, nor should they be interpreted in such a way.

1. An access needle, comprising:
a needle housing;
a needle shaft having a portion disposed within the needle housing;
a lumen within the needle shaft;
an entry port at a proximal end of the needle shaft;
a needle tip at a distal end of the needle shaft; and
an exit port on a sidewall of the needle shaft, wherein the exit port is disposed nearer the distal end of the needle shaft than the proximal end, and wherein the lumen extends from the entry port to the exit port.

2. (canceled)

3. The access needle of claim 1, further comprising:
a guide surface configured to guide a wire from the lumen through the exit port.

4. The access needle of claim 3, further comprising:
a wire in the lumen.

5. The access needle of claim 4, further comprising:
a second wire in the lumen; and
a second entry port at the proximal end of the needle shaft to receive the second wire.

6. (canceled)

7. The access needle of claim 1, further comprising:
a lock for attaching the needle shaft to an endoscope channel of an endoscope.

8. The access needle of claim 7, wherein the needle shaft is adjustable within the needle housing to allow movement of the needle shaft relative to a tip of the endoscope.

9. (canceled)

10. The access needle of claim 1, further comprising:
a sheath disposed around the needle shaft.

11. The access needle of claim 10, further comprising: a locking mechanism configured to lock the needle shaft in position relative to the sheath.

12. The access needle of claim 10, wherein at least one of the needle housing, the sheath, the needle shaft, or the distal end of the needle shaft is rotatable for modification of an exit direction of a wire from the exit port, by rotation of the exit port.

13. The access needle of claim 10, wherein the needle shaft is slidably disposed within the sheath.

14. The access needle of claim 1, wherein the sheath has a proximal end and a distal end, and wherein the distal end tapers toward the needle tip of the needle shaft.

15-16. (canceled)

17. The access needle of claim 1, wherein the entry port further comprises:
an attachment feature for attachment of a syringe to provide fluid into the lumen to the exit port.

18. The access needle of claim 1, wherein the lumen forms a guiding surface to bend the wire to the exit port within the needle shaft.

19. The access needle of claim 1, further comprising:,
one or more markers disposed adjacent to at least one of the needle tip or the exit port, the one or more markers being detectable with an ultrasound radiation or an electromagnetic radiation detector.

20. The access needle of claim 19, further comprising one or more second markers disposed adjacent to at least one of the needle housing, the needle shaft, or a needle handle, the one or more second markers configured to scatter an ultrasound radiation or an electromagnetic radiation.

21. A method, comprising:
placing a needle in a vicinity of an organ, the organ comprising a target portion, wherein the needle comprises: a needle housing, a needle shaft, a lumen within the needle shaft, an entry port at a proximal end of the needle shaft, a needle tip at a distal end of the needle shaft, and an exit port on a sidewall of the needle shaft;
piercing the organ with the needle tip;
injecting a contrast medium into the organ through the lumen in the needle shaft;
adjusting a position of the needle in the organ based on an image of the organ, the image comprising the contrast agent, so that the exit port points to the target portion; and
inserting a wire through the entry port, the lumen, and the exit port, to access the target portion.

22. The method of claim 21, wherein piercing the organ with the needle tip further comprises directing an electric current through the needle tip into an access point in the organ to dilate the access point in the organ.

23. The method of claim 21, wherein adjusting the position of the needle in the organ comprises rotating the needle housing about a longitudinal axis in the needle shaft.

24. The method of claim 21, wherein adjusting the position of the needle in the organ comprises directing a radiation to the organ and collecting a scattered radiation from the organ with an imaging system.

25. The method of claim 21, further comprising removing the needle housing and the needle shaft and guiding a medical device through the wire, to the target portion.

26. The method of claim 21, wherein the target portion is an obstructed duct, further comprising:
removing the needle housing and the needle shaft; and
guiding a stent through the wire to the obstructed duct.

27. The method of claim 21, further comprising:
directing an electric current through the needle tip into an access point in the organ, to cauterize a bleeding.

28. The method of claim 21, wherein adjusting a position of the needle in the organ based on an image of the organ comprises receiving an ultrasound radiation or an electromagnetic radiation scattered from one or more markers disposed adjacent to at least one of the needle tip or the exit port, and including an image of the needle in the image of the organ with an ultrasound radiation detector or an electromagnetic radiation detector.

29. The method of claim 21, wherein adjusting a position of the needle in the organ based on an image of the organ comprises receiving an ultrasound radiation or an electromagnetic radiation scattered from one or more markers disposed adjacent to at least one of the needle housing, the needle shaft, or a needle handle, and including an image of the needle in the image of the organ based on the ultrasound radiation or the electromagnetic radiation.

30. A system, comprising:
an access needle to direct a guiding element to a target portion of an organ, the access needle comprising:
a needle housing;
a needle shaft having a portion disposed within the needle housing;
a lumen within the needle shaft;
an entry port at a proximal end of the needle shaft;
a needle tip at a distal end of the needle shaft; and
an exit port on a sidewall of the needle shaft, wherein the exit port is disposed nearer the distal end of the needle shaft than the proximal end, and wherein the lumen extends from the entry port to the exit port; and
an imaging device to direct the access needle in a vicinity of the organ and puncture an access site in the organ with the needle tip, the imaging device further comprising:
a radiation source configured to direct a radiation to the organ,
a radiation detector configured to detect a scattered radiation from the organ,
a controller, configured to receive a signal from the radiation detector and convert the signal into an image, and
a display, configured to display the image from the controller to direct the needle in the vicinity of the organ.

31-40. (canceled)
